(19) United States
(12) Patent Application Publication
(10) Pub. No.: US 2005/0075552 A1
(43) Pub. Date: Apr. 7, 2005

(54) Device and method for determining the amount of hemoglobin by means of inhalation of a predefined amount of carbon monoxide

(76) Inventors: Walter Schmidt, Bayreuth (DE); Nicole Prommer, Bayreuth (DE)

Correspondence Address: LERNER AND GREENBERG, PA, P O BOX 2480, HOLLYWOOD, FL, 33022-2480, US

(21) Appl. No.: 10/995,675

(22) Filed: Nov. 23, 2004

Related U.S. Application Data
(63) Continuation of application No. PCT/EP03/05411, filed on May 23, 2003.

(30) Foreign Application Priority Data
May 23, 2002 (DE) 102 22 750.0

(51) Int. Cl. A61B 5/00

(52) U.S. Cl. 600/364; 600/322; 600/328; 600/529

(57) ABSTRACT

A device for determining a hemoglobin quantity by inhaling a predetermined quantity of carbon monoxide as a bolus has a mouthpiece connected to a bag that can be filled with oxygen. The bag and the mouthpiece are connected via a connecting piece. The device is provided with a first valve for closing a first passage that links the connecting piece with the mouthpiece and a second valve for closing a second passage that links the connecting piece with the bag.

### CROSS-REFERENCE TO RELATED APPLICATION

This is a continuing application, under 35 U.S.C. § 120, of copending international application No. PCT/EP03/05411, filed May 23, 2003, which designated the United States; this application also claims the priority, under 35 U.S.C. § 119, of German patent application No. 102 22 750.0, filed May 23, 2002; the prior applications are herewith incorporated by reference in their entirety.

### BACKGROUND OF THE INVENTION

1. Field of the Invention

The invention relates to a device and a method for determining the amount of hemoglobin by means of inhalation of a predefined amount of carbon monoxide.

The invention relates generally to methods for determining the amount of hemoglobin in humans. It relates in particular to the carbon monoxide (CO) method. In the CO method, a patient or test subject inhales in a predefined amount of a gas mixture containing CO. The inhaled CO is bound completely to hemoglobin [Hb] contained in the blood. A stable CO-Hb complex forms over the course of several minutes. By determining the concentration of the CO-Hb complex before and after the inhalation of CO, the total amount of hemoglobin in the patient can be calculated. The blood volume can then be determined on the basis of the calculated amount of hemoglobin and the hemoglobin concentration.

In Burge, C. and Skinner, S.: J. Appl. Physiol. 97: 623-631, 1995 and Dingley et al.: Crit Care Med 27: 2435-2441, 1999, a CO method is described in which a blood sample is taken before the inhalation of carbon monoxide and again about 10 to 15 minutes after the start of inhalation. The CO method described assumes that, at the time the second blood sample is taken, a constant CO-Hb value has been established in the blood. The known method requires a relatively long carbon monoxide inhalation of at least 10 minutes and is therefore time-consuming.

Another CO method is known from Hutler et al.: Med. Sci. Sports Exerc. 32: 1024-1027, 2000. In that method too, a blood sample is taken prior to inhalation and the CO-Hb concentration in the blood is determined. Also in that method, there is a relatively long carbon monoxide inhalation period of 10 to 15 minutes. During inhalation, blood samples are taken at intervals of 2 minutes and the CO-Hb concentration is determined. A rising concentration curve is observed which reaches a maximum and then falls. Inhalation is discontinued as soon as the CO-Hb concentration falls. This CO method requires a large number of blood samples. It is time-consuming and inconveniences the patient.

A device called a spirometer is used for inhalation of a predefined amount of CO, and a photometer is used to determine the CO-Hb concentration in the blood. The present invention concerns, inter alia, the spirometer.

A spirometer modified for the CO method is known from Christensen P., Blood Gas News 1994, Vol. 3, No. 1. This is a device in which a mouthpiece is attached to one end of a connection tube, while O₂ is delivered via the other end. CO is injected through a line which is attached to the spirometer and in which a one-way valve is arranged. The CO is introduced into a syringe in a separate procedure prior to each application. After the injection, it mixes with the oxygen present in the spirometer.

In the prior art spirometer, because the filling procedure is effected manually and because of the different temperature and air pressure conditions during filling, the injected amount of CO cannot be set precisely. This leads to measurement inaccuracies. Another disadvantage is that filling of the CO just immediately prior to the test has to be carried out, for safety reasons, under an extractor hood. Such an extractor hood is not present in most hospitals and other medical establishments, with the result that the known spirometer can be operated only at a few locations or establishments. Moreover, it is not possible to check whether the injected CO is taken up completely by the test subject. A distortion of the measurement results caused by incomplete uptake of CO cannot be excluded. A particular disadvantage is that the injected CO mixes with the gas present in the spirometer and, as a consequence, only slow uptake by the patient or test subject is possible. The patient is forced to perform inhalation for an undesirably long period of time.

### SUMMARY OF THE INVENTION

It is accordingly an object of the invention to provide a method and device for determining an amount of hemoglobin by the inhalation of a predetermined amount of carbon monoxide which overcomes the above-mentioned disadvantages of the heretofore-known devices and methods of this general type. It is a particular object to make available a device and a method with which the amount of hemoglobin can be determined simply and inexpensively by means of inhalation of a predefined amount of carbon monoxide. The device should also be as easy to handle as possible. According to another object of the invention, the device ought to be able to be used portably and to operate as precisely and rapidly as possible and be completely safe to use. According to a further object of the invention the device should allow a routine check-up with reduced effort. Furthermore, the burden for the patient shall be kept as low as possible.

With the foregoing and other objects in view there is provided, in accordance with the invention, a device for determining an amount of hemoglobin by way of inhalation of a predefined amount of carbon monoxide. The device comprises:
a mouthpiece;
a bag to be filled with oxygen;
a connection piece connecting the mouthpiece to the bag;
a first valve for selectively closing a first passage between the connection piece and the mouthpiece, and a second valve for selectively closing a second passage between the connection piece and the bag.

In the sense of the present invention under the term “oxygen” there has to be understood a gas containing essentially oxygen. The gas may also be mixed with other gases. However, the gas is substantially free of carbon monoxide.

In a first embodiment, there is provided a further connection for filling the connection piece with the predefined amount of carbon monoxide.

In a second embodiment of the invention, the connection piece is configured as a closed, preferably disposable, container containing the predefined amount of carbon monoxide or it is configured to receive therein a closed, preferably disposable, container containing the predefined amount of carbon monoxide.

In other words, according to the invention, a device for determining the amount of hemoglobin by means of inhalation of a predefined amount of carbon monoxide is proposed, with a mouthpiece which is connected via a connection piece to a bag that can be filled with oxygen, wherein a first valve is provided for closing a first passage connecting the connection piece to the mouthpiece, wherein a second valve is provided for closing a second passage connecting the connection piece to the bag. The connection piece may have an attachment for gastight attachment of a sensor for detecting the carbon monoxide concentration prevailing in the connection piece.

The proposed device is portable and easy to use. The provision of the valves makes it possible initially to set up a predefined amount of carbon monoxide in the connection piece. The first valve, and immediately thereafter the second valve, can then be opened, and the predefined amount of carbon monoxide can be inhaled. It should be noted here that the patient first completely inhales the carbon monoxide, and only thereafter the oxygen contained in the bag. In this way, the inhaled carbon monoxide is distributed within in the alveoli and a great amount of the carbon monoxide gas diffuses into the blood immediately. The carbon monoxide breathing time can be reduced to 2 minutes and therefore the burden placed on the patient can be kept extremely low. The proposed device permits very precise, reproducible and rapid measurement of the amount of hemoglobin and blood volume. It is of simple construction and can mainly be made of disposable components. This permits a particularly inexpensive test procedure and a reduced risk of infection. The second valve makes it possible to fill the bag separately with oxygen without the risk of carbon monoxide present in the connection piece mixing with the oxygen prior to inhalation. Separate filling of the bag with oxygen is also possible.

According to an advantageous embodiment, a filter is provided for filtering gas drawn in through the mouthpiece. The filter can be a component part of the mouthpiece or can be provided in the connection piece. It serves in particular to retain fine particles of dust or dirt during inhalation of the gas contained in the device.

According to a further embodiment, a means for absorbing carbon dioxide can be provided in the connection piece or in the first passage. This avoids a situation where the patient is burdened with an undesirably high concentration of carbon dioxide. With the aforementioned features, it is possible, with multiple ventilation from the device, to prepare a gas for inhalation which is harmless and substantially free of carbon dioxide. The means for absorbing carbon dioxide may be chalk. The filter is expediently arranged between the mouthpiece and the means for absorbing carbon dioxide. This ensures that any dirt particles coming loose from the means for absorbing carbon dioxide are not inhaled.

The mouthpiece can be provided with a first attachment piece designed corresponding to the connection piece, so that the mouthpiece can be applied to the connection piece by means of the first attachment piece. This permits simple replacement of the mouthpiece.

According to a further embodiment, a further, preferably closable attachment is provided on the connection piece for the purpose of filling the connection piece with a predefined amount of carbon monoxide. This makes it possible to fill the connection piece from an external container containing carbon monoxide. The further attachment can be closed by a valve.

However, it can also be closed by an automatically closing rubber membrane.

The bag can be provided with a second attachment piece designed corresponding to the connection piece, so that the bag can be applied to the connection piece by means of the second attachment piece. An attachment which can be closed in a gastight manner can be provided on the connection piece or on the second attachment piece for the purpose of filling the bag with oxygen. This makes it possible to fill the bag with oxygen separately and then to connect the filled bag to the connection piece.

It has proven particularly advantageous if the connection piece is designed as a closed disposable container containing the predefined amount of carbon monoxide. In this way, it is always ensured that the amount of carbon monoxide contained in it is inhaled by the patient. It is then no longer necessary to introduce carbon monoxide by means of an injector syringe. In contrast to the latter situation, it is thus no longer necessary to work under an extractor hood. Errors when introducing the predefined amount of carbon monoxide are also avoided.

It is also possible, however, for the connection piece to be designed in such a way that a closed, preferably disposable, container containing the predefined amount of carbon monoxide can be received in it. In this case, the connection piece can, for example, have a flexible tube section, and the closed, preferably disposable, container containing the carbon monoxide can be formed by a glass or metal ampule. After the ampule has been introduced into the connection piece, it can be broken or opened from the outside, so that the carbon monoxide contained in the ampule escapes into the gastight, closed connection piece.

The first attachment piece and/or second attachment piece and/or the connection piece can be provided with a means for opening the container. The opening means can be designed in such a way that the container can be opened from outside and the carbon monoxide contained in it can be inhaled through the mouthpiece. The opening means can have a spike-shaped opening element. The connection piece can, for example, be cylindrical. It can be connected in a gastight manner to the mouthpiece and the bag by means of the attachment pieces. In the context of the present invention, the term “gastight” is to be understood as meaning that, for at least a period of one hour, only an insignificant amount of CO can escape from the connection piece, so that it is not necessary to take into consideration any impairment of the measurement accuracy. The necessary leaktightness is afforded by familiar materials such as plastic, glass or metal.

If the connection piece is in the form of a cylinder, the opening means can have a closure connectable to the cylinder and movable axially with respect to the cylinder, and also at least one opening element which is arranged in the closure or in the cylinder, projects into the cylinder interior and is used for opening a, preferably disposable, container received in the cylinder. The opening element provided in the closure and/or in the cylinder can in each case be configured like a spike. With the proposed embodiment, it is especially simple to open a container received in the cylinder. By means of an axial displacement of the closure relative to the cylinder, the opening element or opening elements is/are driven through a wall of the, preferably disposable, container. The carbon monoxide contained therein is released into the cylinder through the opening(s) formed. The container may also be provided with at least a vent or valve, respectively, allowing the container to be refilled several times with carbon monoxide. Such a vent may include a closing element which is received in a gastight manner in a seat. For example, the closing element may be in the form of a metal ball which closes a gastight manner a passage being provided through the wall of the container. For this purpose the passage is formed by the seat which is made by a flexible material. For opening of the passage it is just necessary to push the ball through the passage.

The means for absorbing carbon dioxide can be contained in an exchangeable container or in the mouth piece in the attachment piece. The container expediently bears tightly on an inner wall of the connection piece. The container is made gas-permeable in order to permit a flow of gas. As the container lies in a gastight manner on an inner wall of the connection piece or in the mouth piece in the attachment piece, designed for example as a cylinder, or of the closure, an undesired flow of gas past the means for absorbing carbon dioxide is avoided.

According to another embodiment feature, the sensor can be a component part of an arrangement for measuring the concentration of carbon monoxide. For this purpose, the attachment for gastight attachment of the sensor on the connection piece can, for example, be a threaded junction piece.

The arrangement is expediently designed in such a way that it is optionally possible also to measure the concentration of CO in the environment of the mouthpiece. In this way, it is possible to check whether the CO contained in the connection piece has been inhaled completely by the patient. It is also possible to check whether inhaled CO has been undesirably exhaled by the patient through the nose or, possibly, through the mouth and past the sides of the mouthpiece.

The preferably battery-operated arrangement expediently has a means for displaying the CO concentration. The display means can, for example, be an arrangement for indicating concentration. In addition, an alarm signal can be generated. The alarm signal is expediently generated if the measured CO concentration exceeds a predefined value.

The mouthpiece can also be formed in two parts. In this case, a further connection piece can be provided in which the first valve and the attachment for the sensor are provided. This further connection piece can be screwed with one end onto the connection piece. A mouthpiece designed as a disposable part can be fitted onto the other end.

According to a further embodiment feature, an attachment element for attachment of a breathing bag can be provided between the bag and the connection piece or between the mouthpiece and the connection piece. The attachment element can also replace the mouthpiece. The aforementioned features make it possible to use the device also on intubated patients.

According to a further aspect of the invention, a method is provided for determining the amount of hemoglobin by the following steps:
taking a first blood sample and determining a first concentration COHbᵥ of a CO-Hb complex in the first blood sample,
inhaling a predefined amount CO_{geg} of CO for a duration of at most 3 minutes,
taking a second blood sample 3 to 9 minutes after the start of inhalation and determining a second concentration COHbₙ of the CO-Hb complex in the second blood sample,
determining the amount of hemoglobin tHb using the first and second concentrations.

The proposed method makes it possible to determine the amount of hemoglobin simply, quickly, and in a manner which causes the patient minimal inconvenience. The device according to the invention can in particular be used for this purpose. Using such a device ensures that, with the very first inspiration, the predefined amount of CO is drawn into the alveoli and thus almost completely passed into the blood. Consequently, the inhalation can already be terminated after a duration of at most 3 minutes. The second blood sample can be taken just 3 to at most 9 minutes after the start of inhalation and a second concentration COHbₙ of the CO-Hb complex can be determined. Tests have shown that, just 3 to 9 minutes after the start of inhalation, the CO has become uniformly distributed in the blood and the CO-Hb complex has reached its target concentration. The time of taking the second blood sample may be calculated in dependence of a predetermined distribution of CO in the blood.

It has proven expedient to take the second blood sample 3 to 5 minutes, preferably 4 minutes, after the start of inhalation.

According to one embodiment, a third blood sample can be taken 5 to 10 minutes, preferably 6 minutes, after inhalation, and a third concentration of the CO-Hb complex can be determined in the third blood sample. To determine the amount of hemoglobin, in this case a mean concentration formed from the second and third concentrations can be set in relation to the first concentration. This permits particularly exact determination of the amount of hemoglobin.

According to a further embodiment, the amount CO_{R} of CO remaining in the device and in the lungs is measured after inhalation. Also, after inhalation, the exhaled amount CO_{abg} of CO can be measured. This permits an exact determination of the amount of CO taken up by the patient and, consequently, a particularly exact determination of the amount of hemoglobin. For this purpose, the volume of gas remaining in the device and in the lungs can be labeled by addition of a defined amount of helium. The helium can be present, mixed with the carbon monoxide, in the disposable container. Subsequent determination of the helium concentration permits a conclusion regarding the amount of carbon monoxide not taken up by the patient. It has proven expedient to determine the total amount of hemoglobin tHb according to the following equation (see e.g. HEINICKE et al., 2001, Int. J. Sports Med, Vol. 22, 504-12):
tHb=K×(CO_{geg}−CO_{R}−CO_{abg}−CO_{Myo})×100/((COHbₙ−COHbᵥ)×H_{z})
where:
tHb=total amount of hemoglobin,
K=constant (current air pressure, current temperature, thermal expansion factor),
CO_{geg}=amount of CO in the device before inhalation,
CO_{R}=amount of CO in the device and lungs after inhalation,
CO_{abg}=after inhalation, the amount of CO exhaled by the patient being disconnected from the device until the meantime of the second and/or third blood example,
CO_{Myo}=amount of CO diffused from hemoglobin to myoglobine,
COHbᵥ=CO-Hb concentration before inhalation,
COHbₙ=CO-Hb concentration determined between 3 and 9 minutes after start of inhalation,
H_{z}=Hüfner number. The Hüfner number is a number in the range of 1.34 to 1.39.

According to a further advantageous embodiment of the method, the concentration of CO can be measured in the patient's mouth region during inhalation. If a concentration in excess of a predefined value is measured, a signal can be emitted to show that the test is not proceeding in the correct manner. The test is then terminated.

The proposed method is particularly accurate and simple and can be carried out quickly and routinely.

Other features which are considered as characteristic for the invention are set forth in the appended claims.

Although the invention is illustrated and described herein as embodied in a device and method for determining the amount of hemoglobin by means of inhalation of a predefined amount of carbon monoxide, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a diagrammatic sectional side view of a first embodiment according to the invention;

FIG. 2 is a similar view of a second embodiment of the device according to the invention;

FIG. 3 is a similar view of a third embodiment of the device according to the invention; and

FIG. 4 is a diagrammatic sectional side view of a container for carbon monoxide.

### DESCRIPTION OF THE PREFERRED EMBODIMENTS

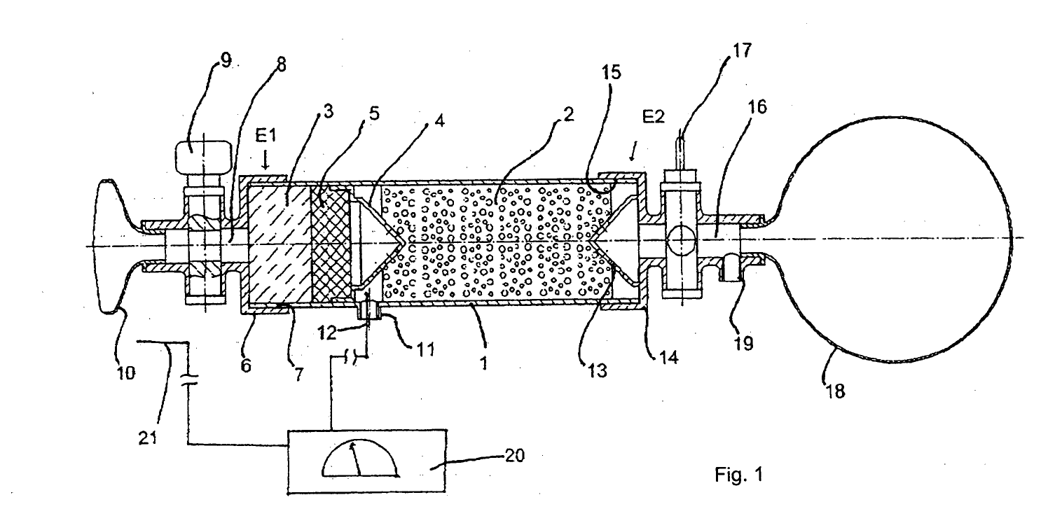

Referring now to the figures of the drawing in detail and first, particularly, to FIG. 1 thereof, a disposable container 2 filled with a predefined amount of CO is received in an axially displaceable manner in a cylindrical connection piece 1 made of plastic, for example. The disposable container 2 is made, for example, of a plastic through which CO does not pass. The disposable container 2 can also be made of metal, for example aluminum. The disposable container 2 expediently lies in a substantially gastight manner against the inner wall of the connection piece 1. This ensures that all the CO contained in the disposable container 2 is taken up by the patient upon the first inhalation or first inspiration. For this purpose, an O-ring, for example, or a peripheral plastic sealing lip (not shown here) can be provided on the inner wall of the connection piece 1.

A filter 3 is provided at a first end E1 of the connection piece 1. A first, conically shaped spike 4 is also provided at an end E1, its tip projecting into the interior space surrounded by the connection piece 1. The first spike 4 is supported on a container 5. It is expediently made of a holed or slotted metal plate or the like. The container 5 is filled with a means that absorbs CO₂, for example with calcium carbonate. The container 5 also expediently lies in a substantially gastight manner against the inner wall of the connection piece 1. For this purpose, a further O-ring seal or a further peripheral plastic sealing lip can be provided on the inner wall of the connection piece 1. Reference number 6 designates a first attachment piece which, by way of a first thread 7, can be connected in a gastight manner to the connection piece 1. The first attachment piece 6 has a first passage 8 which can be closed by means of a first valve 9. The first value 9 may be a 3-way value. A mouthpiece 10, preferably designed as a disposable part, is mounted, preferably detachably, on the free end of the first passage 8. It should be understood that the term “mouthpiece” is used herein to also include a connector for a breathing tube. That is, instead of direct mouth-inhalation, the device may also be used for forced inhalation through a breathing tube. Near the first end E1, the connection piece 1 also has an attachment 11 for gastight attachment of a sensor 12 for detecting the carbon monoxide concentration in the connection piece. The attachment 11 can be a threaded junction piece or the like. A second, conically shaped spike, designated by reference number 13, is mounted on a second attachment piece 14. The second attachment piece 14 can be mounted in a gastight manner on a second end E2 of the connection piece 1 by means of a second thread 15. The second attachment piece 14 has a second passage 16 which can be closed by means of a second valve 17. A bag 18 is mounted at the free end of the second passage 16. A junction piece 19 provided with a one-way valve (not shown here) serves to fill the bag 18 with oxygen. Reference number 20 designates an arrangement which measures CO and to which the sensor 12 is attached. A further sensor 21 for measuring the concentration of carbon monoxide in the area of the mouthpiece 10 is also attached to the arrangement 20.

The attachment pieces 6 and 14 can, like the connection piece 1, be made of plastic. To achieve a gastight connection between the attachment pieces 6, 14 and the connection piece 1, the attachment pieces 6, 14 can be provided with O-rings (not shown here). It is also possible, however, to achieve a tight connection by providing the first 6 and second thread 15 with a joint grease.

The device functions as follows:

The connection piece 1 is supplied with the disposable container 2 received in it, and with the filter 3 and the container 5 as a unit. The disposable container 2 has a closed housing, i.e. the first spike 4 has not yet penetrated this housing. To avoid contamination, the first end E1 and the second end E2 of the connection piece 1 are sealed, for example, with a peel-off metal or plastic foil. Likewise, the attachment 11 can also be sealed off with a plastic membrane or with a metal foil. To make ready the device, the protective foil provided at the first end E1 is firstly removed and the first attachment piece 6 is screwed onto the connection piece 1. The first valve 9 is closed. The protective foil provided at the second end E2 is then removed and the second attachment piece 14 with closed second valve 17 is screwed onto the second end E2 of the connection piece 1. The first 4 and the second spike 13 are thus driven into the cylinder surfaces of the disposable container 2. The carbon monoxide contained in the disposable container 2 escapes into the connection piece 1. The bag 18 is filled with oxygen via the junction piece 19, either before or after the second attachment piece 14 is screwed on. The device is now ready for determining the total amount of hemoglobin.

To carry out the measurement, the patient places the mouthpiece 10 in his mouth. He exhales through the 3-way value 9 into ambient air. Thereafter 3-way value 9 is turned to open the passage to the connection piece 1 and the second valve 17 is opened in succession. The patient now inhales the carbon monoxide contained in the connection piece 1. To ensure that all the carbon monoxide is inhaled by the patient, the latter breathes in and out several times through the connection piece 1. Carbon dioxide (CO₂) exhaled into the connection piece 1 is absorbed by the CO₂-absorbing means in the container 5. The concentration of carbon monoxide in the connection piece 1 can be measured continuously by means of the assembly 20 and the sensor 12 connected to the latter. With the diagrammatically illustrated further sensor 21, it is also possible to directly monitor the concentration of carbon monoxide in the area of the patient's mouth and nasal cavity. If an increase is observed, this points to a lack of leaktightness in the area of the mouthpiece 10. The measurement can in this case be terminated.

In the context of the present invention, it is considered advantageous that the carbon monoxide required for the measurement is made available in the connection piece 1 by way of a disposable container 2. However, it is also possible for the carbon monoxide required for the measurement to be introduced into the connection piece 1 from an external device prior to the measurement. For this purpose, the first valve 9 or the second valve 17 can be designed as three-way valves. The first valve 9 and/or the second valve 17 can in this case be provided with a special connector for filling the connection piece 1 with carbon monoxide. However, it is also possible for the connection piece 1 to be provided with a further separate connector 24 for filling it with carbon monoxide. This connector can, for example, be combined with the attachment 11. In particular, provision of the first valve 9 and of the second valve 17 also ensures, in the case of external filling of the connection piece 1, that the latter, at the start of the measurement, is filled with the predefined amount of carbon monoxide. In this case too, upon the first inhalation, the patient breathes in almost the entire amount of carbon monoxide made available. This advantageously makes the method for determining the total amount of hemoglobin simpler and more rapid.

The method is carried out, for example, in the following way:

A blood sample is taken from the patient prior to inhalation of the carbon monoxide. The concentration COHbₙ of the CO-Hbᵥ complex in the blood is then determined by photometry. Thereafter, using the device according to the invention, the patient inhales, for a duration of at most 3 minutes, preferably 2 minutes, the carbon monoxide present in the connection piece 1. A second blood sample is taken from the patient 4 minutes after the start of inhalation, and a third blood sample is taken 6 minutes after the start of inhalation. The second and third blood samples are used to calculate a mean value COHbₙ of a concentration of the CO-Hbₙ complex. A residual amount of carbon monoxide CO_{R} remaining in the device and in the lungs after inhalation is calculated by means of the sensor 12, and a CO amount CO_{abg} exhaled by the patient is determined also via the sensor 21. From the values calculated, it is possible to determine the total amount of hemoglobin tHb according to the following equation:
tHb=K×(CO_{geg}−CO_{R}−CO_{abg}−CO_{Myo})×100/((COHbₙ−COHbᵥ)×H_{z})
where:
tHb=total amount of hemoglobin,
K=constant (current air pressure, current temperature, thermal expansion factor),
CO_{geg}=amount of CO in the device before inhalation,
CO_{R}=amount of CO in the device and lungs after inhalation,
CO_{abg}=after inhalation, the amount of CO exhaled by the patient being disconnected from the device until the meantime of the second and/or third blood example,
CO_{Myo}=amount of CO diffused from hemoglobin to myoglobine,
COHbᵥ=CO-Hb concentration before inhalation,
COHbₙ=CO-Hb concentration determined between 3 and 9 minutes after start of inhalation,
H_{z}=Hüfner number. The Hüfner number is a number in the range of 1.34 to 1.39.

The carbon monoxide remaining in a device for determining the amount of hemoglobin, in particular the device according to the invention, and in the lungs can be calculated using a predefined amount of helium added to the test gas containing the carbon monoxide. For this purpose, the helium can be made available in the device, for example mixed together with the predefined amount of carbon monoxide.

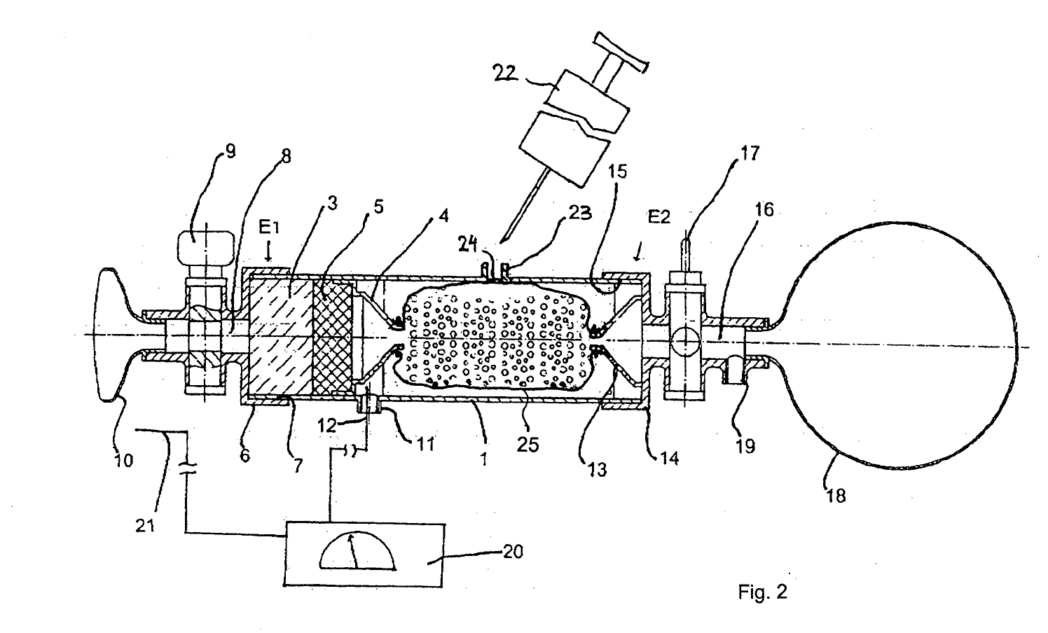

The second embodiment illustrated in FIG. 2 is distinguished from the first embodiment in that the carbon monoxide is introduced not by way of the closed container but by selective injection into the chamber of the connection piece 1. The injection may be effected, by way of example, with a syringe 22 and through a port 23. The port 23 is closed with a self-sealing membrane 24. That is, upon piercing with the needle of the syringe 22, injection of the carbon monoxide, and the withdrawal of the needle, the port 23 is automatically sealed. In another embodiment, the port may be a threaded gas injection port for attachment and injection via a CO charging source. The port 23, furthermore, may be provided with a valve.

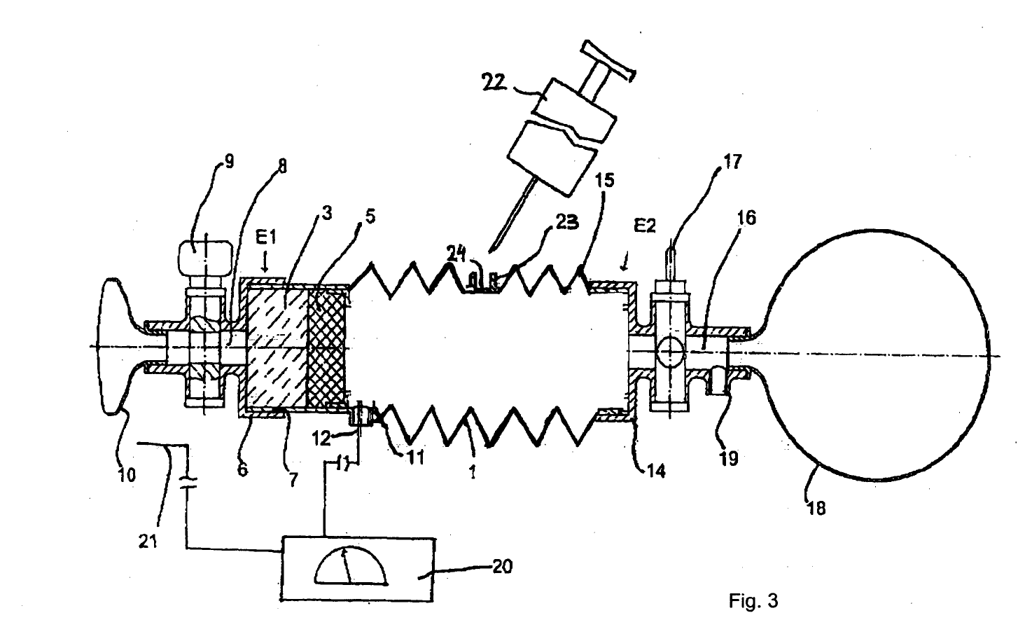

The third embodiment illustrated in FIG. 3 is distinguished from the second embodiment in that the connection piece 1 forms the container for storing carbon monoxide. The connection piece 1 is designed to have a flexible wall. As shown in FIG. 3 the connection piece 1 may be a bellows which is provided—like in the second embodiment—with a port 23 being closed with a self-sealing membrane 24.

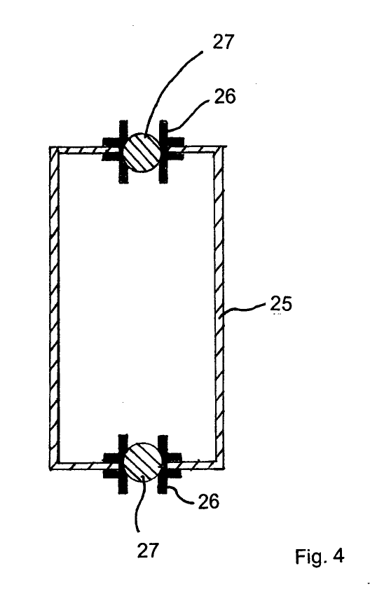

FIG. 4 shows a further embodiment of a container 25 to be taken up for example in the connection piece 1 shown in FIG. 2. In this embodiment the container 25 is made of cylindrical shape. At both ends there is provided a seat 26 in which there is taken up in a gastight manner a metal ball 27. The seat 26 is made for example by rubber and forms a passage through the wall of the container 25. By exerting a external pressure upon the metal ball 27 it is pushed inside the container 25 and the passage is opened. A predetermined amount of carbon monoxide taken up in the container can now be inhaled by a patient. For refilling the container it is possible to remove the seat 26 from the container and then to remove the metal ball from the interior of the container. Subsequently the seat 26 and then the ball 27 can be reassembled e.g. within a glove box containing a predetermined concentration of carbon monoxide.

1. A device for determining an amount of hemoglobin by way of inhalation of a predefined amount of carbon monoxide, the device comprising:
a mouthpiece;
a bag to be filled with oxygen;
a connection piece connecting said mouthpiece to said bag;
a first valve for selectively closing a first passage between said connection piece and said mouthpiece, and a second valve for selectively closing a second passage between said connection piece and said bag; and
a further connection for filling said connection piece with the predefined amount of carbon monoxide.

2. The device according to claim 1, which further comprises a filter disposed for filtering carbon monoxide drawn through said mouthpiece.

3. The device according to claim 1, which further comprises means for absorbing carbon dioxide disposed in one of said connection piece and said first passage.

4. The device according to claim 1, wherein said mouthpiece has a first attachment piece configured in accordance with said connection piece, and said mouthpiece is attached to said connection piece by way of said first attachment piece.

5. The device according to claim 1, wherein said further connection is a selectively closable attachment at said connection piece.

6. The device according to claim 1, wherein said bag includes a second attachment piece configured in accordance with said connection piece, for connecting said bag to said connection piece by way of said second attachment piece.

7. The device according to claim 6, which further comprises an attachment configured for gastight closure and disposed at said connection piece or at said second attachment piece for filling said bag with oxygen.

8. The device according to claim 1, wherein said connection piece has an attachment for gastight attachment of a sensor for detecting a carbon monoxide concentration prevailing in said connection piece.

9. The device according to claim 8, wherein said sensor is a component part of a configuration for measuring the concentration of carbon monoxide.

10. The device according to claim 9, wherein said configuration is enabled to optionally measure a concentration of carbon monoxide in a vicinity of said mouthpiece.

11. The device according to claim 9, wherein said configuration is battery-operated and includes a further CO sensor and a display for displaying the concentration of carbon monoxide.

12. The device according to claim 1, which comprises an attachment element for attachment of a breathing bag disposed between said bag and said connection piece or between said connection piece and said mouthpiece.

13. The device according to claim 1, wherein said mouthpiece is an attachment element for attachment to a breathing tube.

14. A device for determining an amount of hemoglobin by way of inhalation of a predefined amount of carbon monoxide, the device comprising:
a mouthpiece;
a bag to be filled with oxygen;
a connection piece connecting said mouthpiece to said bag;
a first valve for selectively closing a first passage between said connection piece and said mouthpiece, and a second valve for selectively closing a second passage between said connection piece and said bag; and
said connection piece being configured as a closed container containing the predefined amount of carbon monoxide or configured to receive therein a closed container containing the predefined amount of carbon monoxide.

15. The device according to claim 14, which further comprises a device for opening said container functionally associated with at least one of said first attachment piece, said second attachment piece, and said connection piece.

16. The device according to claim 15, wherein said opening device is configure to enable opening said container from outside and to allow the carbon monoxide contained therein to be inhaled through said mouthpiece.

17. The device according to claim 15, wherein said opening device includes a spike-shaped opening element.

18. The device according to claim 14, wherein the container is a disposable container.

19. The device according to claim 15, wherein said sensor is a component part of a configuration for measuring the concentration of carbon monoxide.

20. The device according to claim 19, wherein said configuration is enabled to optionally measure a concentration of carbon monoxide in a vicinity of said mouthpiece.

21. The device according to claim 19, wherein said configuration is battery-operated and includes a further CO sensor and a display for displaying the concentration of carbon monoxide.

22. The device according to claim 15, which comprises an attachment element for attachment of a breathing bag disposed between said bag and said connection piece or between said connection piece and said mouthpiece.

23. The device according to claim 15, wherein said mouthpiece is an attachment element for attachment to a breathing tube.

24. A method for determining an amount of hemoglobin, which comprises the following method steps:
taking a first blood sample and determining a first concentration COHbₙ of a carbon monoxide-hemoglobin CO-Hb complex in the first blood sample;
inhaling a predefined amount CO_{geg} of carbon monoxide CO for a duration of at most 3 minutes;
taking a second blood sample 3 to 9 minutes after a start of inhalation and determining a second concentration COHbₙ of the CO-Hb complex in the second blood sample; and
determining the amount of hemoglobin tHb from the first and second concentrations.

25. The method according to claim 24, which comprises taking the second blood sample 3 to 5 minutes after the start of inhalation.

26. The method according to claim 24, which comprises taking the second blood sample approximately 4 minutes after the start of inhalation.

27. The method according to claim 24, which comprises taking a third blood sample 5 to 10 minutes after the start of inhalation, and determining a third concentration of the CO-Hb complex in the third blood sample.

28. The method according to claim 27, which comprises taking the third blood sample approximately 6 minutes after the start of inhalation.

29. The method according to claim 27, wherein the step of determining the amount of hemoglobin comprises forming a mean concentration from the second and third concentrations and setting the mean concentration in relation to the first concentration.

30. The method according to claim 24, which comprises, subsequent to inhalation, measuring an amount CO_{R} of carbon monoxide remaining in an inhalation device and in the lungs of the subject.

31. The method according to claim 24, which comprises, subsequent to inhalation, measuring an exhaled amount CO_{abg} of carbon monoxide.

32. The method according to claim 24, which comprises determining the amount of hemoglobin tHb according to the following equation:
tHb=K×(CO_{geg}−CO_{R}−CO_{abg}−CO_{Myo})×100/((COHbₙ−COHbᵥ)×H_{z})
where:
tHb=total amount of hemoglobin,
K=constant (current air pressure, current temperature, thermal expansion factor),
CO_{geg}=amount of CO in the device before inhalation,
CO_{R}=amount of CO in the device and lungs after inhalation,
CO_{abg}=after inhalation, the amount of CO exhaled by the patient being disconnected from the device until the meantime of the second and/or third blood example,
CO_{Myo}=amount of CO diffused from hemoglobin to myoglobine,
COHbᵥ=CO-Hb concentration before inhalation,
COHbₙ=CO-Hb concentration determined between 3 and 9 minutes after start of inhalation,
H_{z}=Hüfner number. The Hüfner number is a number in the range of 1.34 to 1.39.

33. The method according to claim 30, wherein the carbon monoxide remaining in the inhalation device and the carbon monoxide remaining in the lungs is calculated with a predefined amount of helium inhaled by means of the device.

34. The method according to claim 24, which comprises inhaling from the device according to claim 1.

35. The method according to claim 24, which comprises inhaling from the device according to claim 14.
